(19) United States
(12) Patent Application Publication
(10) Pub. No.: US 2024/0376060 A1
(43) Pub. Date: Nov. 14, 2024

(54) A SMART COVALENT ORGANIC FRAMEWORK AND A PROCESS FOR CARBON DIOXIDE ADSORPTION INDUCED SWITCHABLE ANTIBACTERIAL ACTIVITY THEREFROM

(71) Applicant: COUNCIL OF SCIENTIFIC AND INDUSTRIAL RESEARCH AN INDIAN REGISTERED BODY INCORPORATED UNDER THE REGN., New Delhi, Delhi (IN)

(72) Inventors: Ajayaghosh AYYAPPANPILLAI, Thiruvananthapuram Kerala (IN); Arindam MAL, Thiruvananthapuram Kerala (IN); Rakesh MISHRA KUMAR, Thiruvananthapuram Kerala (IN); Dileep Kumar BHASKARAN NAIR SARASWATHY AMMA, Thiruvananthapuram Kerala (IN); Jubi JACOB, Thiruvananthapuram Kerala (IN); Sreejith SHANKAR POOPPANAL, Thiruvananthapuram Kerala (IN)

(21) Appl. No.: 18/294,429

(22) PCT Filed: Jan. 6, 2023
(86) PCT No.: PCT/IN2023/050015

§ 371 Date: Feb. 1, 2024

(30) Foreign Application Priority Data
Jan. 6, 2022 (IN) 202211000696

(51) Int. Cl. C07D 259/00

(52) U.S. Cl. CPC C07D 259/00

(57) ABSTRACT

The present invention is related to a class of stable 2D covalent organic frameworks with multiple dimethyl amino groups that can trap carbon dioxide at ambient temperature and pressure, and an economical, environmentally-friendly process for the generation of transient surface charges and subsequent self-exfoliation of the COF into ultrathin nanosheets. The said exfoliated material possess activity against pathogenic bacteria. The invention further discloses a carbon dioxide induced exfoliation process that is completely reversible upon heat treatment, whereby control over bacterial growth is achieved via an efficient antibiotic switch.

### FIELD OF THE INVENTION

The present invention is related to a stable 2D covalent organic frameworks [COFs] with multiple dimethyl amino groups that can trap carbon dioxide at ambient temperature and pressure, and an economical, environmentally-friendly process for the generation of transient surface charges and subsequent self-exfoliation of the COF into ultrathin nanosheets. The said exfoliated material possess activity against pathogenic bacteria. Particularly, present invention relates to a carbon dioxide induced exfoliation process that is completely reversible upon heat treatment, whereby control over bacterial growth is achieved via an efficient antibiotic switch.

### BACKGROUND AND PRIOR ART OF THE INVENTION

Since the development of single layer graphene, research on ultrathin two dimensional (2D) materials has grown enormously and has stretched beyond the realm of graphene (Reference may be made to (a) Zhang, ACS Nano, 2015, 9, 9451; (b) Tan, et al., Chem. Rev., 2017, 117, 6225; (c) M-Balleste, et al., Nanoscale, 2011, 3, 20; (d) Zhuang, et al., Adv. Mater., 2015, 27, 403; (e) Xu, T. Liang, M. Shi, H. Chen, Chem. Rev., 2013, 113, 3766). 2D nanomaterials such as polymer-based nanosheets resemble graphene and exhibit excellent properties such as tunable framework structures, light weight, flexibility, high surface area, and exceptional electronic properties which make them promising substance for next-generation functional materials (Reference may be made to (a) Thomas, Angew. Chem. Int. Ed., 2010, 49, 8328; (b) Colson, et al., Nat. Chem., 2013, 5, 453; (c) Ding, et al., Chem. Soc. Rev., 2013, 42, 548; (d) Huang, et al., Nat. Rev. Mater., 2016, 1, 16068. (e) Yaghi, et al., Science, 2017, 355, eaal1585). The 2D-cocalent organic frameworks can provide extra flexibility in terms of material design with desired properties (Reference may be made to (a) Bunck, et al., J. Am. Chem. Soc., 2013, 135, 14952; (b) Liu, et al., Adv. Mater., 2014, 26, 6912; (c) Sun, et al., Angew. Chem. Int. Ed., 2017, 57, 1034).

Simultaneous with the development of new and improved functional materials, global climate change and excessive CO₂ emissions have caused widespread public concern in recent years. Thus, tremendous efforts have been made towards CO₂ capture and conversion (Reference may be made to (a) Artz, et al., Chem. Rev. 2018, 118, 434. (b) Sanz-Pérez, et al.,Chem. Rev. 2016, 116, 19, 11840). Consequently, the past decade has witnessed a constant rise in developing functional porous materials (Reference may be made to Singh, et al., Chem. Soc. Rev. 2020, 49, 4360) based on reticular chemistry (Reference may be made to Yaghi, J. Am. Chem. Soc. 2016, 48, 15507), where two-dimensional (2D) and three dimensional covalent organic frameworks (COFs) (Reference may be made to (a) Côté, et al., Science 2005, 310, 1166; (b) Côté, et al., J. Am. Chem. Soc. 2007, 129, 12914-12915; (c) Wan, et al., Angew. Chem., Int. Ed. 2009, 48, 5439; (d) Ding, et al., Chem. Soc. Rev. 2013, 42, 548; (e) Colson, et al., Nat. Chem. 2013, 5, 453; (f) Waller, et al., Acc. Chem. Res. 2015, 48, 3053-3063) have captured a great deal of attention due to its tailor-made architectures. However, efficient exfoliation of COFs and presence of CO₂ responsive groups are required for this purpose.

Several methods have been used by different research groups across the globe

to induce exfoliation of covalent organic frameworks into 2D nanosheets. A boronate ester linked COF has been reported by the condensation reaction between 2,3,6,7,10,11-hexahydroxytriphenylene and 1,3,5-tris[4-phenylboronic acid]benzene and ultrasound sonication exfoliated the bulk COF and produced multilayered 2D COF nanostructures (Reference may be made to Berlanga, et al., Small, 2011, 7, 1207). Mechanical grinding induced delamination of chemically stable porous COFs synthesized using a solvothermal condensation reaction between 2,4,6-triformylphloroglucinol and various diamines was also shown to produce covalent organic nanosheets (Reference may be made to Chandra, et al., J. Am. Chem. Soc., 2013, 135, 17853). Chemical exfoliation of an anthracene-based COF synthesized by a solvothermal condensation reaction between 2,6-diaminoanthracene and 2,4,6-triformylphloroglucinol also resulted in exfoliated nanosheets (Reference may be made to Khayum, et al., Angew. Chem. Int. Ed., 2016, 55, 15604). Such exfoliated nanosheets of COFs find applications as charge carriers (Reference may be made to Sun, et al.,J. Phys. Chem. C., 2016, 120, 14706) and anode material for Li-ion batteries (Reference may be made to (a) Haldar, et al., Adv. Energy Mater., 2018, 8, 1702170; (b) Wang, et al., J. Am. Chem. Soc., 2017, 139, 4258).

A majority of the existing exfoliation methods suffer from non-homogeneity over the thickness of the sheets, energy expensive methods, chemical modification leading to change in properties or destruction of the material, use of environmentally harmful strategies, etc. Hence there is an unmet need to produce a safe, environment-friendly and energy efficient protocol for the exfoliation of COFs into ultra-thin nanosheets.

By introducing suitable CO₂ responsive moieties in 2D COFs, not only CO₂ gas can be stored, but also effective conversion or utlilisation can be achieved for much desired antimicrobial properties without generating substantial antibiotic resitance. Tertiary amines, in water, react with CO₂ to form hydrogen carbonate and quaternary ammonium species which reverts to neutral amines upon heating in the presence of an inert gas such as argon (Reference may be made to (a) Lee, et al., Chem. Commun., 2015, 51, 2036; (b) Guo, et al., Adv. Mater., 2012, 3, 584; (c) Che, et al., Angew. Chem. Int. Ed., 2015, 54, 8934). Several reports have educated the storage of carbon dioxide in COFs (Reference may be made to: (a) Babaroa, et al., Exceptionally high CO₂ storage in covalent-organic frameworks: Atomistic simulation study, Energy Environ. Sci., 2008,1, 139-143; (b) Ozedemir, et al. Covalent Organic Frameworks for the Capture, Fixation, or Reduction of CO₂, Front. Energy Res., 2019, 7, 77; (c) Zeng, et al. Covalent Organic Frameworks for CO₂ Capture, Adv. Mater., 2016, 28, 2855-2873; (d) Furukawa, et al. Storage of Hydrogen, Methane, and Carbon Dioxide in Highly Porous Covalent Organic Frameworks for Clean Energy Applications, J. Am. Chem. Soc. 2009, 131, 8875-8883). Several COFs have also been reported to possess antibacterial activity (Reference may be made to: (a) Wan, et al., Microporous Frameworks as Promising Platforms for Antibacterial Strategies Against Oral Diseases, Front. Bioeng. Biotechnol., 2020, 8, 628; (b) Bhunia, et al., 2D Covalent Organic Frameworks for Biomedical Applications, Adv. Funct. Mater., 2020, 30, 2002046).

Several patents have educated the synthesis and applications of COFs. US 20140148596A1 (Dichtel, et al.) disclosed the synthesis of crystalline COFs comprising a phthalocyanine moiety and a boron-containing multifunctional linking group joined by boronate ester bonds with application in electronic materials. WO 2014/057504 A1 (Banerjee, et al.) reported COFs synthesized via mechanochemical method/solvo-thermal method and with the help of mechanical grinding that exhibited stability towards acidic, basic and neutral conditions and their delamination into few layer covalent organic nano sheets. Chemically stable hollow spherical covalent organic framework having mesoporous walls with high surface area, a process for synthesis of the same and its adsorption capability has been disclosed by U.S. Pat. No. 10,266,634 (Banerjee, et al.). U.S. 20190161623 disclosed a method for modifying COFs comprising an imine group using phenylacetylene for converting the imine group into a quinoline group, and their superhydrophobic properties.

2,5-bis (2-(dimethylamino)ethoxy)terephthalohydrazide based tertiary amine containing COFs and their carbon dioxide sorption properties has been reported, however, these sorption properties are evaluated in the solid state without self-exfoliation. These COFs are also not shown to possess any antibacterial activity (Reference may be made to Gottschling, et al., Molecular Insights into Carbon Dioxide Sorption in Hydrazone-Based Covalent Organic Frameworks with Tertiary Amine Moieties, Chem. Mater. 2019, 31, 1946-1955). 2D COFs constructed from building blocks having tertiary amines functional units have with adequate CO₂ adsorption capacity simultaneous with reversible exfoliation and switchable antibacterial activity is not found in the literature. Such COFs can provide an ideal “antibiotic switch on/off” based platform by generating CO₂ mediated transient charges, which can be again neutralized by removing the CO₂ gas from the medium.

Upon extensive investigations related to the exfoliation of porous organic frameworks, the inventors found that a majority of the existing exfoliation methods suffer from non-homogeneity over the thickness of the sheets, energy expensive methods, chemical modification leading to change in properties or destruction of the material, use of environmentally harmful strategies, etc. Hence there is an unmet need to produce a safe, environment-friendly and energy efficient protocol for the exfoliation of COFs into ultra-thin nanosheets.

### OBJECTIVES OF THE INVENTION

Main objective of the present invention is to the design and development of an economical and environmentally-friendly process for the self-exfoliation of the 2D covalent organic frameworks using reversible adsorption of carbon dioxide.

Another objective is to generate transient surface charges in the COF architecture with multiple dimethyl amino groups, that can trap carbon dioxide at ambient temperature and pressure, leading to subsequent self-exfoliation of the COFs into ultra-thin nanosheets by trapping carbon dioxide under moist conditions.

Yet another objective of the present invention is to provide a smart material with tunable antibacterial activity against pathogenic bacteria, wherein carbon dioxide induced exfoliation process is completely reversible upon heat treatment, and control over bacterial growth is achieved via an efficient antibiotic switch.

Yet another object of the present invention to provide functional group access within the COFs for interaction with carbon dioxide leading to generation of ionic groups on their surface, whereby the said process allows subsequent self exfoliation of the COFs into ultra-thin nanosheets.

### SUMMARY OF THE INVENTION

Present invention provides a process for the exfoliation of porous organic compounds, specifically covalent organic frameworks (COFs), using carbon dioxide in presence of water, moisture or humidity, wherein a minimum of 0.4 mmol/g capture of carbon dioxide is effected under ambient conditions at room temperature and atmospheric pressure, whereby a smart and switchable antibacterial activity is induced.

Accordingly, present invention provides two dimensional, porous, crystalline, stable covalent organic frameworks (COFs) of formula I

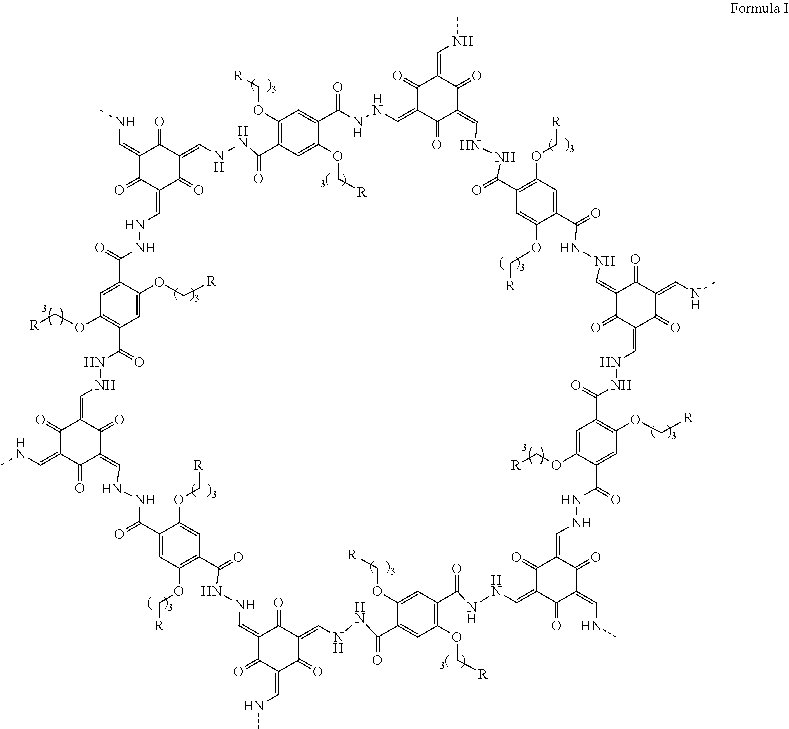

wherein R=NMe₂ or Et.

In yet another embodiment of the present invention, the said COFs comprise of a single or plurality of dimethylamino groups.

In yet another embodiment, present invention provides a process for the preparation of covalent organic frameworks (COFs) of formula I as disclosed herein, wherein said process comprising the steps of:
i. synthesizing hydrazide of formula A using intermediate of formula 2, 3, and 5;

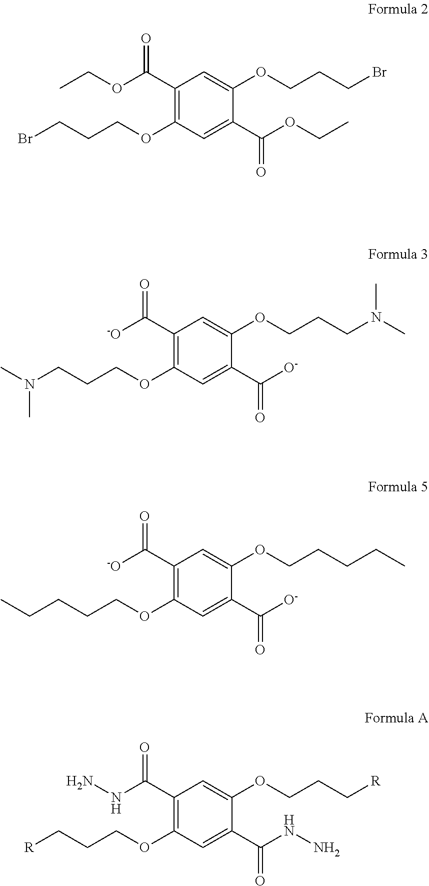

wherein R=NMe₂ or Et;
ii. charging a Teflon-lined steel bomb with 1 equivalent of aldehyde and 1.5 equivalents of the hydrazide of formula A as obtained in step (i) to obtain a mixture;
iii. adding mesitylene, 1,4-dioxane and acetic acid in to the mixture as obtained in step (ii) to obtain a mixture;
iv. sonicating the mixture as obtained in step (iii) for a period in the range of 5 to 10 min to get a homogenous dispersion;
v. sealing the teflon-lined steel bomb and heating at temperature in the range of 100 to 120° C. for three days;
vi. collecting the precipitate by centrifugation at speed in the range of 500 to 1500 rpm for a period in the range of 1 to 10 min;
vii. washing the precipitate as obtained in step (vi) with water;
viii. purification by Soxhlet extraction using acetone, tetrahydrofuran, and methanol;
ix. drying the powder at temperature in the range of 50 to 100° C. under vacuum for a period in the range of 1 to 24 h to obtain covalent organic frameworks (COFs) of formula I.

In yet another embodiment of the present invention, the aldehyde used is selected from aromatic tri-aldehydes, preferably 2,4,6-triformylphloroglucinol.

In yet another embodiment of the present invention, the hydrazide of formula A used is selected from aromatic terephthalohydrazides preferably 2,5-bis (3-dimethylamino)propoxyterephthalohydrazide or 2,5-bis(pentyloxy)terephthalohydrazide.

In yet another embodiment, the present invention provides a process for the reversible self-exfoliation of covalent organic frameworks (COFs) as disclosed herein, comprising the steps of:
i. dispersing or suspending COFs in water, at a w/v ratio in the range of 1-10 mg/mL to obtain a dispersion;
ii. purging the dispersion as obtained in step (i) with a balloon filled with carbon dioxide at a temperature in the range of 25-32° C. at atmospheric pressure for a period in the range of 10 to 30 min to obtain exfoliated ultra-thin nanosheets, whereby ionic charges are created on the COF surface, and the zeta potential of the said COFs is >20 mV, preferably >30 mV.

In yet another embodiment of the present invention, the creation of ionic surface charges leads to self-exfoliation or delamination of the said COFs into ultra-thin nanosheets.

In yet another embodiment of the present invention, the thickness of the nanosheets is uniform and <5 nm, preferably <2 nm, and the ultra-thin nanosheets are comprised of a maximum of single-or bi-layer of the COFs.

In yet another embodiment of the present invention, the said process is specific to a combination of COFs comprising of a single or plurality of dimethylamino groups and carbon dioxide, whereby the use of other gases, inter alia, argon, nitrogen, hydrogen, etc. does not result in either or all of the induction of ionic charges or self-exfoliation or delamination of the COFs with or without dimethylamino groups.

In yet another embodiment of the present invention, carbon dioxide is adsorbed onto the delaminated COF, that is thermally reversible via mild heat treatment at a temperature in the range 30-50° C. in presence of argon for a period in the range of 5 to 10 min, whereby neutralization of surface charges is effected via the elimination of adsorbed carbon dioxide, and the zeta potential values return to a near zero value and the initial multi-layer morphology of the COF is reinstated.

In yet another embodiment of the present invention, the self-exfoliated ultra-thin nanosheets of the COFs possess tunable antibacterial activity against both gram positive and gram negative bacteria, with >60% reduction in bacterial growth within 30 minutes of incubating with the exfoliated COFs (1.0 mg/mL), >90% reduction in bacterial growth within 120 minutes of incubating with the exfoliated COFs (1.0 mg/mL), further the said anti-bacterial activity of the COFs is reversible upon heat treatment with <30% reduction in bacterial growth under identical conditions.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE FIGURES

FIG. 1 illustrates the molecular structures of COFs 1 and 2, in accordance with an embodiment of the present disclosure.

FIG. 2 illustrates the packing model for COF-1, in accordance with an embodiment of the present disclosure.

FIG. 3 illustrates the packing model for COF-2, in accordance with an embodiment of the present disclosure.

FIG. 4 illustrates the experimental and predicted XRD [X-Ray Diffraction] patterns of COF-1, confirming an eclipsed packing model as shown in FIG. 2, in accordance with an embodiment of the present disclosure.

FIG. 5 illustrates the experimental and predicted XRD patterns of COF-2, confirming an eclipsed packing model as shown in FIG. 3, in accordance with an embodiment of the present disclosure.

FIG. 6 illustrates the thermogravimetric analysis of COFs 1 and 2, confirming thermal stability at least until 350° C., in accordance with an embodiment of the present disclosure.

FIG. 7 illustrates the FT-IR [Fourier Transform-Infra Red] spectra of COF-1, in accordance with an embodiment of the present disclosure.

FIG. 8 illustrates the FT-IR spectra of COF-2, in accordance with an embodiment of the present disclosure.

FIG. 9 illustrates the ¹³C CP-MAS [Cross Polarization—Magic Angle Spinning] NMR [Nuclear Magnetic Resonance] spectra of COF-1 (bottom) and COF-2 (top), in accordance with an embodiment of the present disclosure.

FIG. 10 illustrates the CO₂ adsorption profile of COF-1 at 303 K, confirming 0.4 mmol/g adsorption capacity, in accordance with an embodiment of the present disclosure.

FIG. 11 illustrates the carbon dioxide induced reaction of tertiary amine group of COF-1 and the formation of surface charges, in accordance with an embodiment of the present disclosure.

FIG. 12 illustrates zeta potential diagram of exfoliated COF-1 after purging with CO₂ showing the induction of surface ionic charges, in accordance with an embodiment of the present disclosure.

FIG. 13 illustrates the transmission electron micrographs of the exfoliation process of COF-1 upon carbon dioxide purging showing a few-layered morphology of ultra-thin nanosheets, in accordance with an embodiment of the present disclosure.

FIG. 14 illustrates the transmission electron micrographs of one to two layer morphologies of COF-1 nanosheets obtained by the exfoliation of COF-1 upon carbon dioxide purging, in accordance with an embodiment of the present disclosure.

FIG. 15 illustrates the (a), (b) atomic force micrographs of the ultra-thin nanosheets obtained by the exfoliation of COF-1 upon carbon dioxide purging. (c), (d) Height profiles of the corresponding white lines drawn on (a) and (b) showing the thickness of 1 nm indicating few-layered structures, in accordance with an embodiment of the present disclosure.

FIG. 16 illustrates the Raman spectra of exfoliated ultra-thin nanosheets of COF-1 deposited on the ITO surface in the presence of gold, showing the presence of bicarbonate ions, confirming the mechanism of exfoliation as suggested in FIG. 22, in accordance with an embodiment of the present disclosure.

FIG. 17 illustrates the experimental set up for CO₂ purging experiment (left) and the zoomed view of circled portion (right), in accordance with an embodiment of the present disclosure.

FIG. 18 illustrates the Tyndall effect of exfoliated ultra-thin nanosheets of COF-1, in accordance with an embodiment of the present disclosure

FIG. 19 illustrates the (a) daylight picture of COF-2 kept in water before CO₂ purging and (b) the absence of any Tyndall effect after CO₂ purging, confirming negligible exfoliation happening in this case, in accordance with an embodiment of the present disclosure.

FIG. 20 illustrates the reversible induction of surface ionic charges in COF-1, wherein the exfoliated COF shows a zeta potential >32 mV and the heated/argon treated exfoliated COF assembles back into the original COF with a near zero zeta potential, in accordance with an embodiment of the present disclosure.

FIG. 21 illustrates the TEM images showing the reversible exfoliation of COF-1, wherein the heated/argon treated exfoliated COF reassembles back into the original COF with a multi-layer structure, in accordance with an embodiment of the present disclosure.

FIG. 22 illustrates the antibacterial activity (E.coli) of carbon dioxide alone, original COF-1 and carbon dioxide treated exfoliated COF nanosheets bearing ionic surface charges, in accordance with an embodiment of the present disclosure.

FIG. 23 illustrates the photographs showing the CFUs [Colony Forming Units] of E.coli after 120 minutes of treatment with (A) control, (B) carbon dioxide only and (c) exfoliated ultra-thin nanosheets of COF-1, in accordance with an embodiment of the present disclosure.

FIG. 24 illustrates the photographs showing the CFUs of E.coli after 30 and 60 min. treatment under various conditions, in accordance with an embodiment of the present disclosure.

FIG. 25 illustrates the antibacterial activity carbon dioxide treated exfoliated COF nanosheets bearing ionic surface charges and its reversibility upon heat/argon treatment, thereby providing access to a smart antibiotic switch for E.coli, in accordance with an embodiment of the present disclosure.

FIG. 26 illustrates the antibacterial activity carbon dioxide treated exfoliated COF nanosheets bearing ionic surface charges and its reversibility upon heat/argon treatment, thereby providing access to a smart antibiotic switch for E.coli, wherein an ideal “antibiotic switch on/off” based platform is demonstrated, in accordance with an embodiment of the present disclosure.

FIG. 27 illustrates the antibacterial activity carbon dioxide treated exfoliated COF nanosheets bearing ionic surface charges and its reversibility upon heat/argon treatment, thereby providing access to a smart antibiotic switch for E.coli. (A) Control,

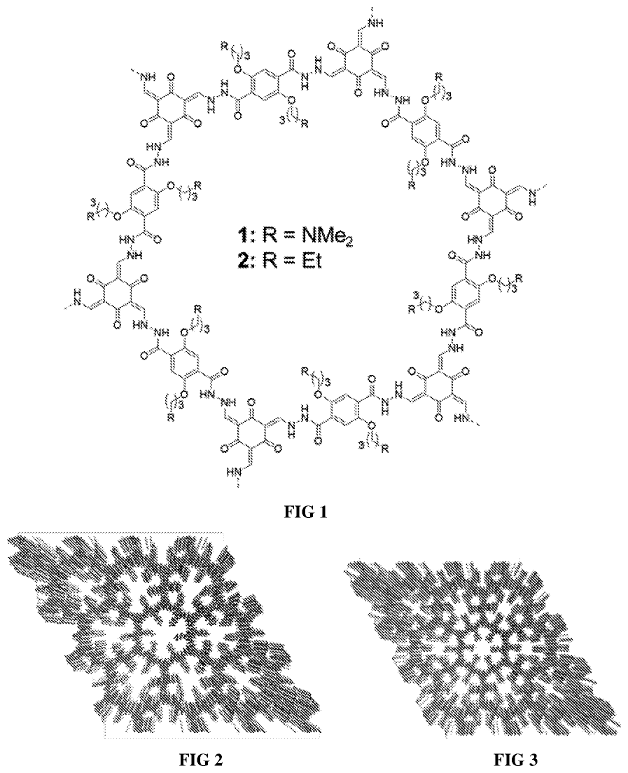

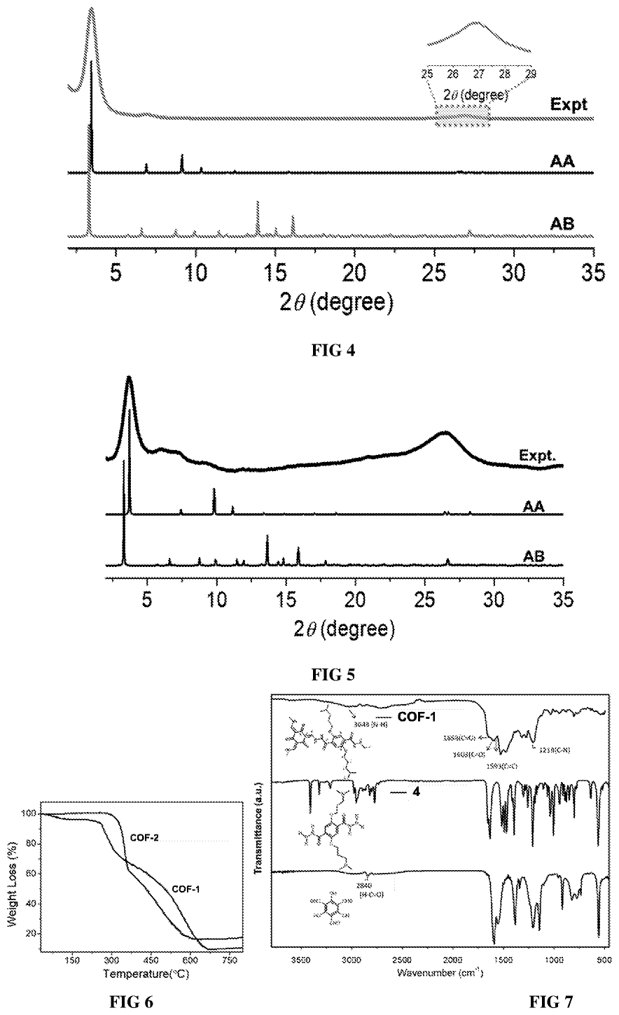

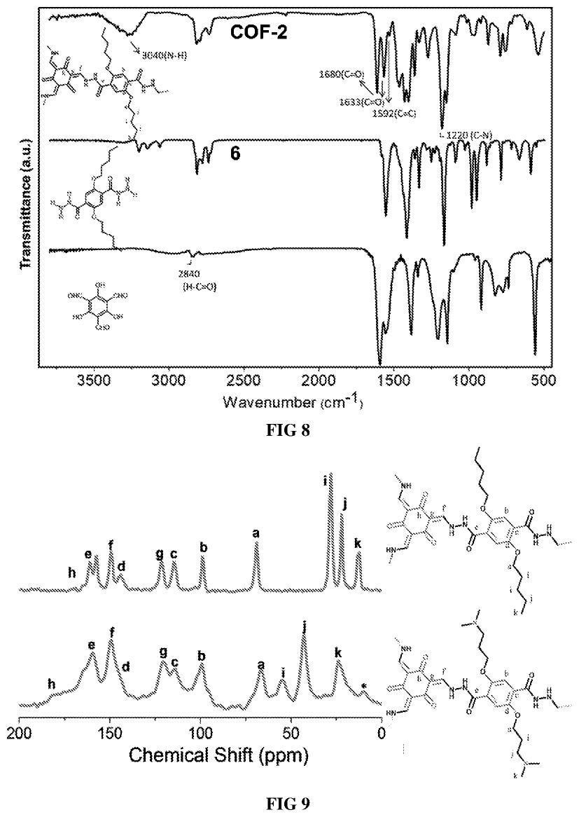

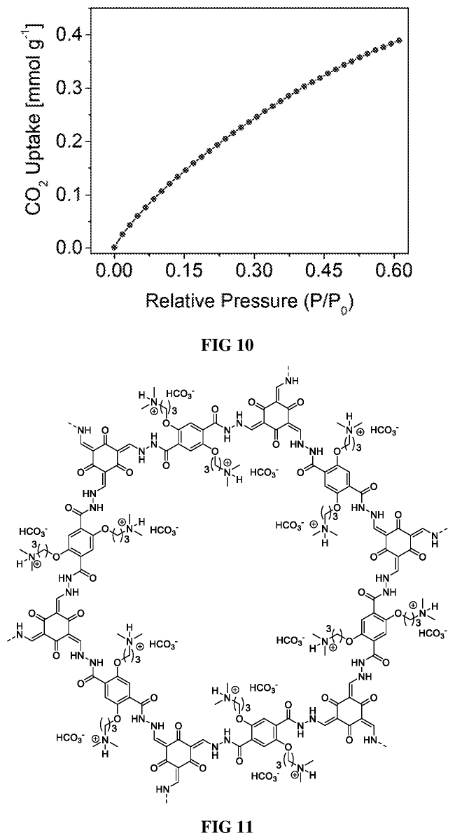

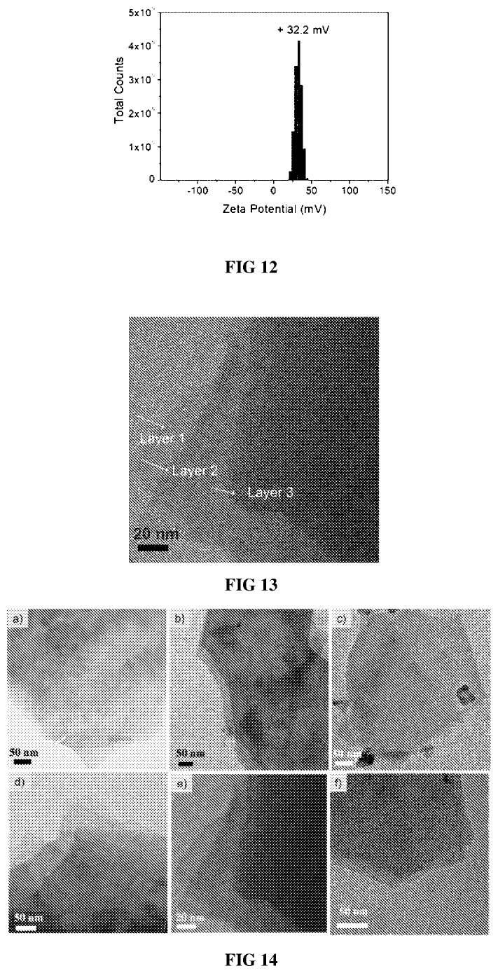

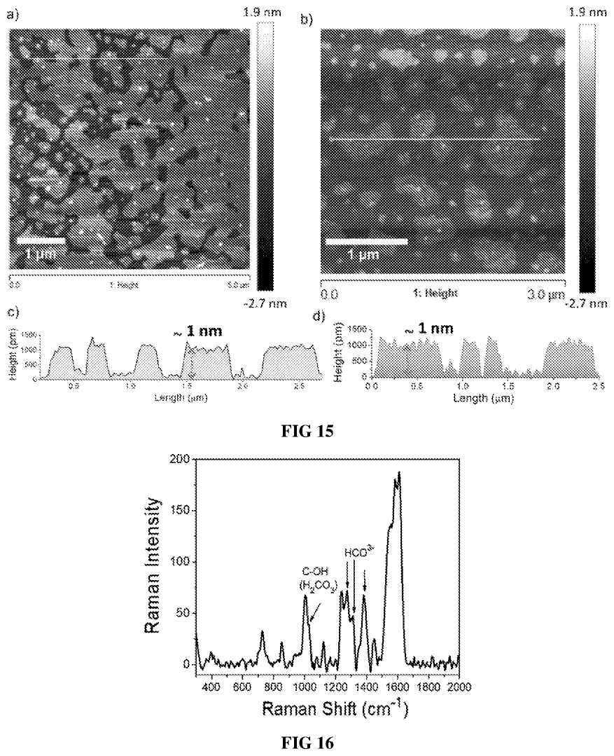

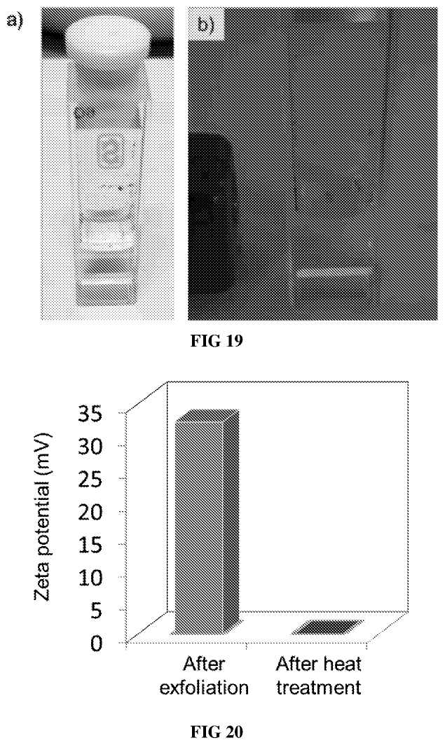

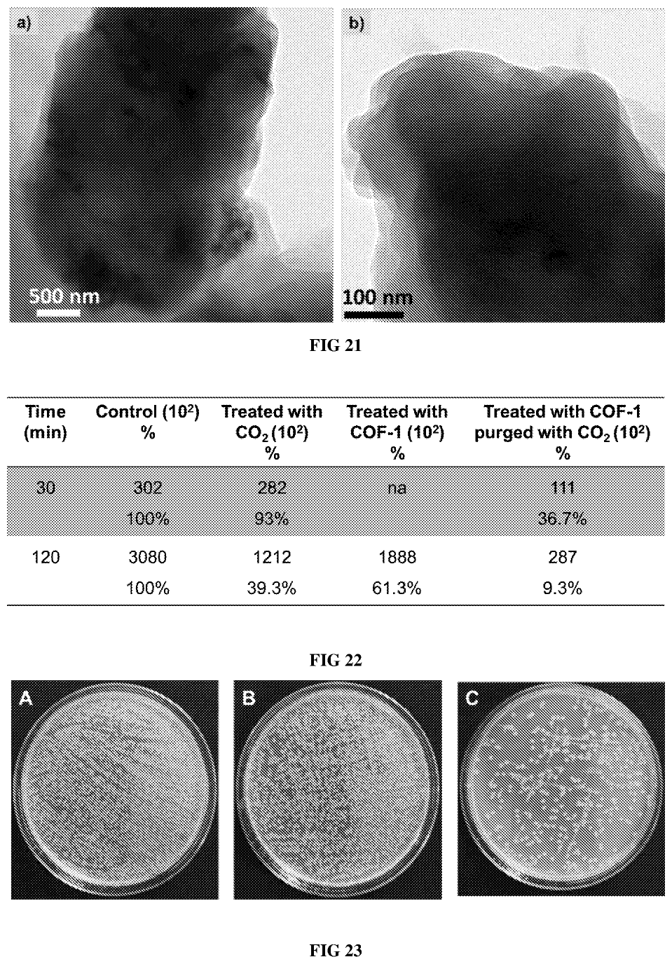

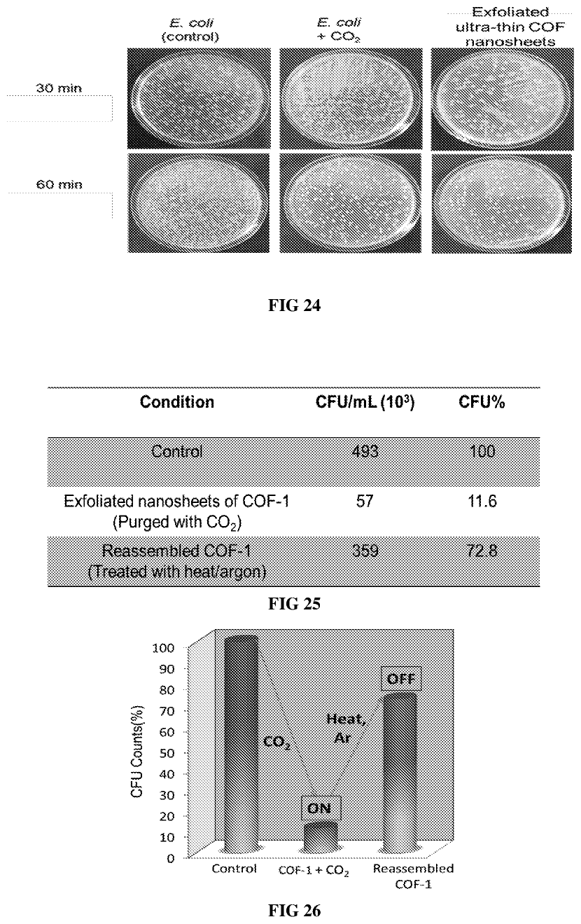

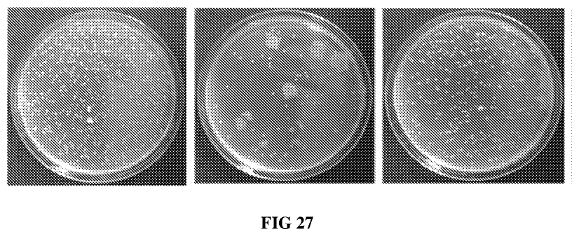

(B) Exfoliated nanosheets of COF-1 obtained by purging with CO₂ and (C) Reassembled COF-1 obtained by treating with heat/argon, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The present invention provides an economical and environmentally-friendly process for the self-exfoliation of the 2D covalent organic frameworks with multiple dimethyl amino groups that can trap carbon dioxide at ambient temperature and pressure.

Further present invention provides functional group access within the COFs for interaction with carbon dioxide leading to generation of ionic groups on their surface, whereby the said process allows subsequent self exfoliation of the COFs into ultra-thin nanosheets.

The invention also intends to provide antibacterial smart COFs, with tunable antibacterial activity, wherein carbon dioxide induced exfoliation is completely reversible upon application of temperature, and bacterial growth is modulated via a smart and efficient antibiotic switch.

The present invention intends to offer a process for the reversible self-exfoliation of porous organic compounds, specifically covalent organic frameworks (COFs), using carbon dioxide in presence of water, moisture or humidity, under ambient conditions, wherein the said COFs comprise of a single of plurality of dimethylamino groups.

The other vital constitutional element in the present invention is the creation of ionic charges on the COF surface, when dispersed or suspended in water and purged with a balloon filled with carbon dioxide under ambient conditions for 10-30 min, such that the zeta potential of the said COFs is >30 mV, leading to the self-exfoliation or delamination of the said COFs into ultra-thin nanosheets of uniform thickness <2 nm and the ultra-thin nanosheets are comprised of a maximum of single-or bi-layer of the COFs.

The said process is specific to a combination of COFs comprising of a single or plurality of dimethylamino groups and carbon dioxide, and other gases such as argon, nitrogen, hydrogen, etc. does not result in the induction of ionic charges or self-exfoliation or delamination of the COFs.

Carbon dioxide adsorption onto the delaminated COF is thermally reversible via mild heat treatment at a temperature in the range 30-50° C. for 5-10 min in presence of argon, whereby neutralization of surface charges is effected via the elimination of adsorbed carbon dioxide, and the zeta potential values return to a near zero value and the initial multi-layer morphology of the COF is reinstated, thereby the self-exfoliated ultra-thin nanosheets of the COFs possess tunable antibacterial activity against both gram positive and gram negative bacteria, further the said anti-bacterial activity of the COFs is reversible upon heat treatment.

Another significant aspect of the present invention discloses the application in domestic, industrial or automobile coatings and personal protection equipments inter alia air filters, membranes, etc. for continuous air purification using reversible carbon dioxide sorption and switchable antibacterial activity, wherein adsorption of carbon dioxide in presence of water, moisture or humidity turns on the antibacterial activity, further increase in temperature above room temperature resets the process leading to ejection of carbon dioxide and reduced antibacterial activity, whereby the material is switched to a set mode for another cycle of carbon dioxide adsorption and resultant antibacterial action.

Although the subject matter has been described in considerable detail with reference to certain examples and implementations thereof, other implementations are possible.

### EXAMPLES

### Example 1

Synthesis of diethyl 2,5-bis(3-bromopropoxy)benzene-1,4-dicarboxylate [2]

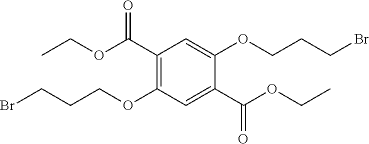 [SMILES: CCOC(=O)c1cc(OCCCBr)c(C(=O)OCC)cc1OCCCBr]

1,3-Dibromopropane (4.38 mL, 10 mmol), TBAB (0.1 g, 0.31 mmol) and K₂CO₃ (2.16 g, 15.6 mmol) were taken in a 250 mL two-neck round bottom flask containing 40 mL dry acetone. The mixture was stirred at room temperature (25-32° C.) for 30 minutes and 5-dihydroxybenzene-1,4-dicarboxylate 1 (2.04 g, 8 mmol) was added dropwise. The reaction mixture was refluxed at 80° C. for 24 h. After cooling the reaction mixture to room temperature (25-32° C.), the solvent was evaporated under reduced pressure. The residue thus obtained was extracted using chloroform, washed with water, brine and dried over anhydrous sodium sulphate. The crude product was subjected to column chromatography (60% chloroform/hexane) over silica gel that gave the pure product. Yield: 85%; ¹H NMR (500 MHZ, CDCl₃): δ=7.26 (s, 2H), 4.39-4.35 (m, 4H), 4.17-4.15 (t, 4H), 3.67-3.65 (t, 4H), 2.36-2.31 (m, 4H), 1.41-1.38 (m, 6H) ppm; ESI-MS (m/z): [M+Na]⁺ Calculated for C₁₈H₂₄Br₂O₆, 516.994; found, 516.993.

### Example 2

Synthesis of 2,5-bis[3-(dimethylamino)propoxy]benzene-1,4-dicarboxylate [3]

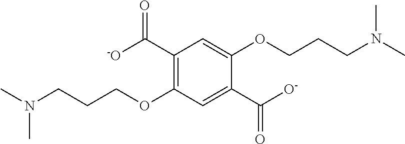

Compound 2 (500 mg, 4.8 mmol) was taken in a 100 mL round bottom flask and 10 mL of dimethylamine solution (2.0 M) in THF was added to it. The reaction mixture was heated to reflux at 80° C. for 12 h. After cooling the reaction mixture to room temperature, the solvent was evaporated. The residue was extracted using chloroform. The organic layer was washed with water, brine, dried over anhydrous sodium sulphate and the solvent was evaporated under reduced pressure to get the crude product. This crude product was used for next reactions without further purification. Yield: 80%; ¹H NMR (500 MHz, MeOD): δ=7.66 (s, 2H), 4.45-4.41 (m, 4H), 4.35-4.33 (t, 4H), 3.51-3.49 (t, 4H), 3.04-3.03 (s, 12H), 2.36-2.31 (m, 4H), 1.43-1.40 (t, 6H) ppm; ESI-MS (m/z): [M+Na]⁺ Calculated for C₂₂H₃₆N₂O₆, 448.257; found, 448.268.

### Example 3

Synthesis of 2,5-bis(3-dimethylamino) propoxyterephthalohydrazide [4]

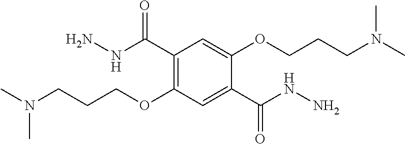

Compound 3 (330 mg, 4.8 mmol) and hydrazine hydrate (2 mL, 9.32 mmol) were taken in a 100 mL two-neck round bottom flask containing 20 mL of dry ethanol. The reaction mixture was refluxed at 80° C. for 12 h. After cooling to room temperature, the product was kept for precipitation. The white precipitate was collected by filtration, washed with water and dried under vacuum to get 0.264 g (Yield: 80%) of a white solid. m.p.: 148-152° C.; ¹H NMR (500 MHz, DMSO-d₆): δ=9.57 (s, 2H), 7.44 (s, 2H), 4.59 (m, 4H), 4.11-4.08 (t, 3H), 2.50-2.49 (m, 12H), 2.39-2.36 (t, 4H), 1.90-1.85 (m, 4H) ppm; ¹³C NMR (125 MHZ, CDCl₃): δ=168.75, 154.96, 129.74, 119.82, 79.23, 61.56, 60.08, 90.31, 31.36 ppm; ESI-MS (m/z): [M+Na]⁺ Calculated For C₁₈H₃₂N₆O₄, 419.248; found, 419.254.

### Example 4

Synthesis of 2,5-dipentoxybenzene-1,4-dicarboxylate [5]

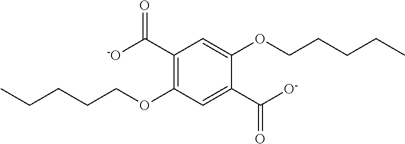

1-Bromopentane (1.5 mL, 8.1 mmol) and K₂CO₃ (2.16 g, 15.6 mmol) were taken in a 250 mL two-neck round bottom flask containing 40 mL dry acetonitrile. The mixture was stirred at room temperature [25° C.] for 30 minutes and 5-dihydroxybenzene-1,4-dicarboxylate 1, (2.04 g, 8 mmol) was added dropwise. The reaction mixture was allowed to refluxe at 80° C. for 24 h. After cooling the reaction mixture to room temperature, the solvent was evaporated under reduced pressure. The residue thus obtained was extracted using chloroform, washed with water, brine and dried over anhydrous sodium sulphate. The crude product was subjected to column chromatography (60% chloroform/hexane) over silica gel that gave the pure product. Yield: 85%; ¹H NMR (500 MHz, CDCl₃): δ=7.26 (s, 2H), 4.29-4.25 (q, 4H), 3.92-3.90 (t, 4H), 1.72-1.68 (m, 4H), 1.38-1.34 (m, 4H), 1.30-1.26 (m, 10H) ppm, 0.84-0.81 (t, 6H); ESI-MS (m/z): [M] Calculated for C₂₂H₃₄O₆, 394.223; found, 394.228.

### Example 5

Synthesis of 2,5-bis(pentyloxy)terephthalohydrazide [6]

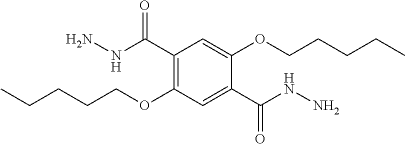 [SMILES: CCCCCOc1cc(C(=O)NN)c(OCCCCC)cc1C(=O)NN]

Compound 5 (1 g, 2.534 mmol) and hydrazine hydrate (8 mL, 253 mmol) were taken in a 100 mL two-neck round bottom flask containing 40 mL dry ethanol. The reaction mixture was refluxed at 80° C. for 12 h. After cooling to room temperature, the product precipitated upon keeping the reaction mixture. The white precipitate formed was collected by filtration, washed with water and dried under vacuum to give 0.856 g (Yield: 85%) of a white solid. m.p.: 139-143° C.; ¹H NMR (500 MHz, DMSO-d₆): δ=9.28 (s, 2H), 7.39 (s, 2H), 4.58 (m, 4H), 4.07-4.05 (t, 3H), 3.47-3.42 (m, 4H), 1.40-1.34 (m, 8H), 0.92-0.87 (t, 6H) ppm; ¹³C NMR (125 MHz, DMSO-d₆): δ=169.09, 154.95, 130.16, 119.93, 74.35, 61.24, 45.32-44.32, 33.41-32.81, 2.06, 23.76, 19.11 ppm; ESI-MS (m/z): [M+H]⁺ Calculated For C₁₈H₃₀N₄O₄, 366.226; found, 367.233.

### Example 6

### Synthesis of COF-1 (R=NMe₂)

A Teflon-lined steel bomb was charged with 2,4,6-triformylphloroglucinol (63 mg, 0.3 mmol), 4 (178.416 mg, 0.45 mmol), 1.5 mL of mesitylene, 1.5 mL of 1,4-dioxane and 0.5 mL of 6 M aqueous acetic acid. This mixture was sonicated for 10 min to get a homogenous dispersion. The Teflon-lined steel bomb was then sealed off and was heated at 120° C. for three days. A brown colored precipitate was collected by centrifugation and washed repeatedly with double-distilled water. The powder collected was then purified by Soxhlet extraction with a series of solvents such as acetone, tetrahydrofuran, and methanol. The obtained solid was then dried at 100° C. under vacuum for 24 h to afford COF-1 as a deep brown colored powder in 85% isolated yield. FT-IR (KBr): νₘₐₓ=3040 (w), 1658 (m), 1603 (m), 1593 (w), 1534 (s), 1486 (s), 1434 (s), 1327 (m), 1284 (m), 1213 (m), 1131 (w), 1041 (w), 962 (w), 899 (w), 804 (w), 776 (w) cm⁻¹; ¹³C CP-MAS NMR (100.61 MHz, solid-state): δ=181.44, 159.79, 149.36, 120.765, 114.12, 99.51, 66.73, 54.85, 43.01 and 24.18 ppm.

### Example 7

### Synthesis of COF-2 (R=Et)

A Teflon-lined steel bomb was charged with 2,4,6-triformylphloroglucinol (63 mg, 0.3 mmol), 6 (158.139 mg, 0.45 mmol), 1.5 mL of mesitylene, 1.5 mL of 1,4-dioxane and 0.5 mL of 6M aqueous acetic acid. This mixture was sonicated for 10 min to get a homogenous dispersion. The Teflon-lined steel bomb was then sealed off and was heated at 120° C. for three days. A light yellow colored was collected by centrifugation and washed repeatedly with double-distilled water. The powder collected was then purified by Soxhlet extraction with a series of solvents such as acetone, tetrahydrofuran, and methanol. The obtained solid was then dried at 100° C. under vacuum for 24 h to afford COF-2 as a light yellow colored powder in 80% isolated yield. FT-IR (KBr): νₘₐₓ=3410 (w), 2956 (w), 2930 (w), 2867 (w), 1680 (s), 1633 (s), 1592 (w), 1537 (s), 1521 (s), 1485 (s), 1456 (s), 1414 (m), 1385 (w), 1320 (m), 2214 (s), 1188 (s), 1126 (w), 1006 (w), 899 (w) 810 (w), 771 (w) cm⁻¹; ¹³C CP-MAS NMR (100.61 MHz, solid-state): δ=170.01, 160.98, 157.46, 149.01, 144.12, 121.46, 114.47, 98.80, 69.15, 27.66, 21.72 and 12.34 ppm.

### Example 8

Carbon dioxide purging experiments and self-exfoliation of COFs

COFs 1 and 2 (2 mg) were suspended in DI water (4 mL) and CO₂ was purged using a CO₂ filled balloon for 30 min. After 30 min. of purging, Tyndall effect was observed for COF-1. Similar purging with other gases such as Ar, N₂ did not show any Tyndall phenomenon demonstrating the specificity of COF-1 towards CO₂. Tyndall effect shown by COF-1 in water indicates the presence of exfoliated ultra-thin nanosheets that were confirmed by morphological analyses. COF-2 did not show any Tyndall effect under similar conditions, confirming the role of N(Me)₂ groups in the exfoliation process.

### Example 9

Antibacterial Studies using Exfoliated COFs

A single colony of E. coli and S. aureus from a nutrient agar (NA) plate was transferred to 10 mL nutrient medium and was grown at 37° C. for 24 h. Bacteria were then harvested by centrifuging at 8000 rpm for 5 min and washed twice with phosphate buffered saline (PBS, pH=7.2±0.2). The supernatant was discarded and the remaining bacterial cells were re-suspended in PBS, and was diluted to an optical density of 1.0 at 600 nm (OD₆₀₀=1.0). The bacteria then were incubated with the COFs (purged with carbon dioxide) at 37°° C. for 16 h in dark. After incubation, all the bacterial suspensions were serially diluted 1×10⁸ fold with PBS. 100 μL from the bacterial dilution was streaked on the NA plates and the colonies formed after 24 h incubation at 37° C. were counted as colony-forming units (CFUs). The bacterial solution without any treatment served as control. The experiment was conducted in triplicates.

### Advantages of the Invention

Economic and environment friendly process for self-exfoliation of COFs
Use of carbon dioxide, an inexpensive reagent
Highly reversible process
Tunable anti-bacterial activity
Presence of ionic charges that can be generated in situ
Applicability in antibacterial coatings, PPE, etc.

I/we claim:

1. Two dimensional, porous, crystalline, stable covalent organic frameworks (COFs) of formula I 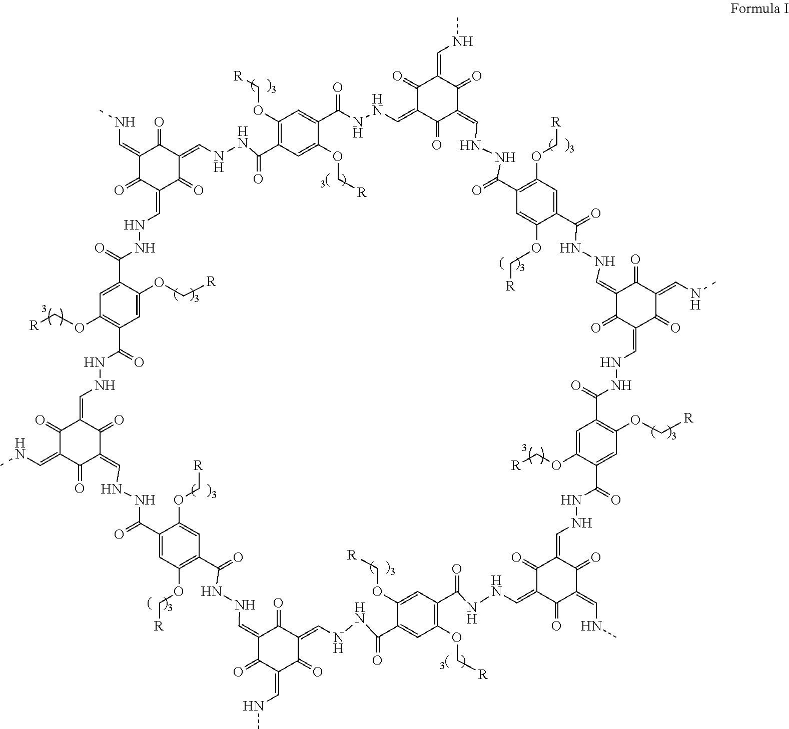
wherein R=NMe₂ or Et.

2. The covalent organic frameworks (COFs) as claimed in claim 1, wherein said COFs comprises a single or plurality of dimethylamino groups.

3. A process for the preparation of covalent organic frameworks (COFs) of formula I as claimed in claim 1, wherein said process comprising the steps of:
i.synthesizing hydrazide of formula A using intermediate of formula 2, 3, and 5; 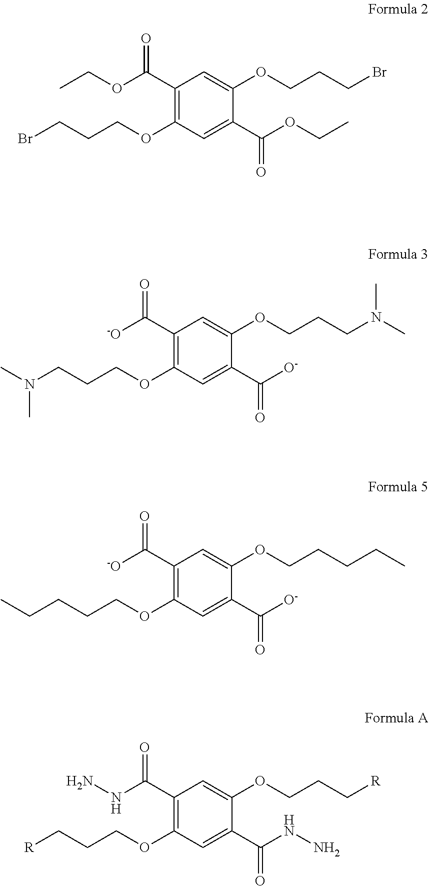
wherein R=NMe₂ or Et;
ii. charging a Teflon-lined steel bomb with 1 equivalent of aldehyde and 1.5 equivalents of the hydrazide of formula A as obtained in step (i) to obtain a mixture;
iii. adding mesitylene, 1,4-dioxane and acetic acid into the mixture as obtained in step (ii) to obtain a mixture;
iv. sonicating the mixture as obtained in step (iii) for a period in the range of 5 to 10 min to get a homogenous dispersion;
v. sealing the teflon-lined steel bomb and heating at temperature in the range of 100 to 120° C. for three days;
vi. collecting the precipitate by centrifugation at speed in the range of 500 to 1500 rpm for a period in the range of 1 to 10 min;
vii. washing the precipitate as obtained in step (vi) with water;
viii. purification by Soxhlet extraction using acetone, tetrahydrofuran, and methanol; and
ix. drying the powder at temperature in the range of 50 to 100° C. under vacuum for a period in the range of 1 to 24 h to obtain covalent organic frameworks (COFs) of formula I.

4. The process as claimed in claim 3, wherein aldehyde used is selected from aromatic tri-aldehydes, preferably 2,4,6-triformylphloroglucinol.

5. The process as claimed in claim 3, wherein hydrazide of formula A used is selected from aromatic terephthalohydrazides, preferably 2,5-bis(3-dimethylamino)propoxyterephthalohydrazide or 2,5-bis(pentyloxy)terephthalohydrazide.

6. A process for the reversible self-exfoliation of covalent organic frameworks (COFs) as claimed in claim 1 comprising the steps of:
i. dispersing or suspending COFs in water, at a w/v ratio in the range of 1-10 mg/mL to obtain a dispersion;
ii. purging the dispersion as obtained in step (i) with a balloon filled with carbon dioxide at a temperature in the range of 25-32° C. at atmospheric pressure for a period in the range of 10 to 30 min to obtain exfoliated ultra-thin nanosheets, whereby ionic charges are created on the COF surface, and the zeta potential of the said COFs is >20 mV, preferably >30 mV.

7. The process as claimed in claim 6, wherein the creation of ionic surface charges leads to self-exfoliation or delamination of the said COFs into ultra-thin nanosheets.

8. The process as claimed in claim 6, wherein the thickness of the nanosheet is uniform and <5 nm, preferably <2 nm, and the ultra-thin nanosheets are comprised of a maximum of single-or bi-layer of the COFs.

9. The process as claimed in claim 6, wherein the said process is specific to a combination of COFs comprising of a single or plurality of dimethylamino groups and carbon dioxide, whereby the use of other gases, inter alia, argon, nitrogen, hydrogen, etc. does not result in either or all of the induction of ionic charges or self-exfoliation or delamination of the COFs with or without dimethylamino groups.

10. The process as claimed in claim 6, wherein carbon dioxide is adsorbed onto the delaminated COF, that is thermally reversible via mild heat treatment at a temperature in the range 30-50° C. in presence of argon for a period in the range of 5 to 10 min, whereby neutralization of surface charges is effected via the elimination of adsorbed carbon dioxide, and the zeta potential values return to a near zero value and the initial multi-layer morphology of the COF is reinstated.
